(19) United States
(12) Patent Application Publication
(10) Pub. No.: US 2003/0135891 A1
(43) Pub. Date: Jul. 17, 2003

(54) Method of transforming intact plants

(75) Inventors: Jean H. Gould, College Station, TX (US); Ronald J. Newton, Greenville, NC (US)

Correspondence Address: LOCKE LIDDELL & SAPP LLP, 600 TRAVIS, 3400 CHASE TOWER, HOUSTON, TX, 77002-3095, US

(73) Assignee: The Texas A&M University System

(21) Appl. No.: 10/304,441

(22) Filed: Nov. 26, 2002

Related U.S. Application Data
(60) Provisional application No. 60/336,809, filed on Dec. 4, 2001.

(51) Int. Cl. A01H 1/00; C12N 15/87; A01H 7/00

(52) U.S. Cl. 800/294; 800/319

(57) ABSTRACT

The present invention provides a method for transforming plants with genetic material. The new method described herein represents a significant improvement in transformation technology. Direct inoculation of an intact pre-formed plant, advantageously without excising the shoot, allows for plant regeneration and makes the procedure genotype-independent while greatly accelerating plant generation by reducing time in tissue culture. Further, the potential for somaclonal variation caused by tissue de-differentiation in culture is greatly reduced. In one embodiment, the invention provides a genotype-independent, direct shoot-based transformation method using Agrobacterium to facilitate recovery of transformed, transgenic plants and to allow transformation of elite germplasm. This technique can be used, for example, with P. taeda L. as described herein. In one embodiment, the transformed shoots can be subjected to antibiotic selection during seedling development.

### RELATED APPLICATION

This application claims the benefit under 35 U.S.C. §119(e) of U.S. Provisional Patent Application Ser. No. 60/336,809 filed Dec. 4, 2001, entitled “Method for Transforming Pines and Other Plants,” incorporated by reference herein.

### GOVERNMENT SUPPORT

This research was supported in part by funds from the USDA Plant Genome Program (Grant No. 93-37300-8859) and the Texas Advanced Technology Program (Grant No. 99990-2219 and/or Grant No. 02-219). Accordingly, the Government has certain rights in the invention.

### SPECIFICATION

### FIELD OF THE INVENTION

This invention is in the field of plant transformation techniques for generating genetically modified plants. More particularly, this invention is in the field of bacterial-mediated transformation techniques for use in conifers and other plants.

### BACKGROUND OF THE INVENTION

Forest products constitute an important cash crop in the United States and around the world. Of the forest species, conifers are the most important. The single most important factor blocking improvement of conifer genotypes through genetic engineering has been a historical inability to regenerate plants following transformation. Significant improvement can be achieved through marker-aided selection and propagation of superior types. Specific and dramatic gains in growth, tolerance to herbicides, environmental stresses, as well as alteration in flowering and in the properties of wood, are now possible in conifers through genetic engineering. However, this achievement may be gained at the cost of genetic diversity, since most plant transformation and regeneration procedures available are limited to regenerable genotypes, reducing diversity in the genetic base of the populations produced. Conifers readily sustain genetic transformations mediated by Agrobacterium tumefaciens (Sederoff et al. 1986; Huang et al. 1994; Wenck et al. 1999). However, reports concerning the recovery of intact plants and of the stable genomic integration of transferred genes in conifers are rare. More generally, callus-based plant transformation techniques, such as through somatic embryogenesis, limit the number of genotypes amenable to plant regeneration following genetic manipulation (U.S. Pat. No. 5,164,310).

Conifer transformation can be accomplished using particle bombardment with subsequent plant regeneration achieved through callus or somatic embryogenesis. Particle bombardment yields transgenic plants that contain multiple copies of the transferred genes. Gene fragmentation and silencing is common in angiosperms transformed through bombardment (Dong et al. 1996; Svitashev et al. 2000). Plant regeneration through somatic embryogenesis is highly genotype-specific and in angiosperms is associated with permanent genetic mutations known as somaclonal variation (Murashige 1974; Li et al. 1989; Hirochika et al. 1996). In cereals, these heritable mutations have been found to adversely impact the agronomic characteristics of subsequent generations of plants (Oard 1996). In comparison, Agrobacterium-mediated transformations are known to produce low copy gene transfers (Dong et al. 1996; Wenck et al. 1999). Also, regeneration of plants from cuttings and shoots has been used in the commercial clonal propagation of plants and trees in the industry for many decades. Regeneration of plants from shoot apical meristems has been used to recover virus-free plant germplasm (Morel et al. 1968).

The use of shoots in micro-propagation is known to carry a low mutation rate and to produce plants that are true to type, if not superior, to the original due to elimination of cryptic virus infection (Morel et al. 1968; Murashige 1974). While bombardment and regeneration through somatic embryogenesis yield acceptable rates of transformation and plant regeneration in easily regenerated conifers, i.e., P. radiata (Walter et al. 1998) and P. abies (Ellis et al. 1989), this approach has been less successful with loblolly pine (P. taeda L.) (Wenck et al. 1999).

To overcome the problems in plant regeneration from callus or embryogenesis following transformation, we previously used A. tumefaciens inoculation of excised shoot apex, in vitro tissue culture selection and regeneration of transformed apex cells, followed by reculturing in a rooting medium (Gould et al. 1991). This original method requires tissue culture manipulation that necessitates highly specialized technicians to execute (Gould et al. 1991; U.S. Pat. No. 5,164,310) and fell short of commercialization goals. Thus, there is a need for methods that avoid some or all tissue culture steps necessary in this and other currently available procedures.

There is a need for an alternative transformation methodology for the production of transgenic plants. Such a need is met by the present invention by providing an in situ transformation method that is applicable to whole plants, such as whole trees. The prior art methods generally require a tissue culture step, and in situ modification of whole plants has not been contemplated hitherto (Gould et al. 1991; U.S. Pat. No. 5,164,310). Other methods of whole plant transformation involve soaking or vacuum infiltration, for example in the case of Arabidopsis thaliana seeds, to generate transgenic plants. Such methods are inefficient and therefore are suitable only for small rapidly growing annuals where one may overcome poor transformation efficiency by growing large numbers of plants and selecting transformants from relatively large populations.

Thus, there is a need for an efficient plant transformation technique capable of practical use in all plants, but especially plants with longer life cycles such as conifers, that permits transformation of plants in situ and that does not require extensive periods in tissue culture.

### SUMMARY OF THE INVENTION

The present invention provides method, apparatus, and system for transforming plants with genetic material. In an advantageous embodiment, the plants are woody plants such as pines. The new method described herein builds on a previous procedure and represents a significant improvement in transformation technology. Direct inoculation of a pre-formed plant shoot in situ allows for direct plant regeneration and makes the procedure genotype-independent. Further, the potential for somaclonal variation caused by tissue de-differentiation in culture is virtually eliminated.

In one embodiment, the invention provides a genotype-independent, shoot-based transformation method using Agrobacterium to facilitate recovery of transformed, transgenic plants and to allow transformation of elite germplasm. This technique can be used, for example, with P. taeda L. as described herein. Shoots of 4-6 week old germinated seeds can be inoculated in situ with A. tumefaciens and the transformed shoots can be subjected to antibiotic selection during seedling development.

In another embodiment, mature shoot meristems are inoculated with A. tumefaciens and transgenic branches are generated. As an example, the generation of transgenic branches and seeds therefrom can reduce the time between transformation and seed collection in woody perennial species by years. In another embodiment, a bud on a juvenile growth or branch is transformed. In another embodiment, a bud on a mature or adult plant growth or branch is transformed. In yet another embodiment, a flower bud is transformed.

In one embodiment, the transformed plant may come from any gymnosperm genera. In a preferred embodiment, the transformed plant may come from any woody genera, particularly from forest trees and fruit trees such as coffee (Cofea spp.), coconut (Cocos nucifera), cassava (Manihot esculenta), peanut (Arachis hypogea), citrus trees (Citrus spp.), cocoa (Theobroma cacao), tea (Camellia sinensis), banana (Musa spp.), avocado (Persea americana), fig (Ficus, casica), guava (Psidium guajava), mango (Mangifera indica), olive (Olea eiropea), papaya (Carica papaya), cashew (Anacardium occidentale), macadamia (Macadamia integrifolia), almond (Prunus amygdalus), cotton (Gossypium hirsutum), rose (Rosaceae), grape vines, and other gymnosperms such as conifers, pines (for example Afghan pine, radiata pine, Virginia pine and Loblolly pine), poplars and eucalyptus.). In a second alternative embodiment, the transformed plant may come from any dicot genera such as canola (Brassica napus, Brassica rapa ssp.), sunflower (Helianthus annuus), soybean (Glycine max.), tobacco (Nicotiana tabacum), potato (Solanum tuberosum), sweet potato (Ipomoea batatus), pineapple (Ananas comosus), sugar beets (beta vulgaris), vegetables, and ornamentals. Examples of vegetable types include brasssica veegtables such as cabbage, broccoli, cauliflower, lettuce, endive, pea, potato, and beans. Beans include guar, locust bean, fenugreek, soybean, garden beans, cowpea, mungbean, lima bean, fava bean, lentils, chickpea, etc. In a third alternative embodiment, the transformed plant may come from any monocot genera such as grain plants, for example, corn, wheat, barley, rice, sorghum and rye.

Shoot-based transformation and regeneration methods of the invention offer a genotype-independent approach to plant transformation that is not considered possible with callus-based methods, or by somatic embryogenesis, and can be used with seedlings identified through marker-aided selection. It is believed that the addition of improved Agrobacterium virulence, direct inoculation of seedlings and effective rooting protocols improves the recovery of transgenic plants such as pines and other conifers.

### BRIEF DESCRIPTION OF THE DRAWINGS

A more particular description of the invention, briefly summarized above, may be had by reference to the embodiments thereof which are illustrated in the appended drawings and described herein. It is to be noted, however, that the appended drawings illustrate only some embodiments of the invention and are therefore not to be considered limiting of its scope, because the invention may admit to other equally effective embodiments.

FIG. 1 is a graphical representation a germinated pine seedling and partially illustrates the practice of an embodiment of the disclosed method.

FIG. 2 shows that seedlings were successfully transformed according to one embodiment of the present invention as indicated by PCR amplification data of markers unique to transformed seedlings which are absent in nontransformed seedlings.

FIG. 3 shows a Southern blot of putative transgenic loblolly pine DNA hybridized with uidA probe indicating that the transferred genes were incorporated into high molecular weight DNA characteristic of stable transformation.

FIG. 4 shows PCR amplification data of the plant/T-DNA border junction indicating that the transferred genes were incorporated into the plant's DNA.

### DETAILED DESCRIPTION OF A PREFERRED EMBODIMENT

The discussion and examples which follow detail the best known method for performing the invention. It will be recognized that variations of this method may include different culture media or different vectors dependent upon the target plant species and the traits to be transferred into the target plants. Pine species were chosen as the target plants in the following example; however, the method outlined below is adaptable to other plants capable of Agrobacterium or other bacterial infection without significant experimentation or deviation from the spirit and scope of this invention.

One factor blocking improvement of loblolly and other commercial pine genotypes through genetic engineering has been lengthy regeneration periods that heretofore are necessary in bacterial-mediated transformation procedures. To overcome this block in plant regeneration, the present inventors developed a direct inoculation procedure using Agrobacterium that profoundly reduces the period that plant tissue is cultured and avoids the potential for somaclonal variation caused by tissue de-differentiation in culture (Hirochika, 1996). The method exploits virulent Agrobacteriuim tumefaciens (Hood et al. 1986; 1990) and the transformation competent cells present in the shoot apex and apical meristem. Transgenic plants of loblolly pine (P. taeda) and putative transgenic plants of afghan (P. eldarica), radiata (P. radiata) and Virginia (P. virginia) pines have been recovered using this method.

An exemplary method of the present invention is shown in Table 1. Briefly, seedlings of the plant species of interest, such as that of the genus Pinus, are germinated. Germination for three to four weeks is usually adequate but the actual periods that are required for effective transformation differ depending upon the plant species.

The germinated seedlings are directly inoculated in situ with a virulent strain of A. tumefaciens harboring genetic sequences for transfer into plants. This novel direct inoculation of seedlings virtually eliminates the time and labor burden typical of bacterial mediated transformations. The bacteria is grown in the presence of appropriate antibiotics to select those cells that are carrying the gene of interest in the presence of appropriate inducers of bacterial virulence and hormonal inducers of cell division in the shoot apex. Seedling shoots are wounded and inoculated with A. tumefaciens and the entire seedling is returned to culture. The bacteria and the seedlings are co-cultured for a period sufficient to permit transformation.

Optionally, the inoculated seedlings are placed on media containing antibiotics that will remove the A. tumefaciens infection and select for transgenic tissues.

Total germination, selection and recovery periods may vary for seeds or seedlings of various plant species but for conifer seedlings the total time can be as short as five weeks.

“Intact”, when referring to plant material, generally describes plant tissue that is not detached from a plant specimen that would otherwise form two or more separate independent plant specimens fully capable of independent manipulation.

“Shoot apex”, in reference to this invention, refers generally to the apical region of a plant shoot. The apical region is understood to include, but not being limited to, the apical meristem, leaf primordia, or subtending elongating leaves, or a combination thereof.

### EXAMPLES

### Plant Material

Open pollinated P. taeda L. seeds were obtained from Forestry Suppliers, Inc. (Jackson, Miss.). The seeds were surface sterilized according to the method of Chang et al. (1991). The sterilized seeds were then aseptically germinated in 8% water/agar dispensed in sterile plastic petri dishes and cultured under a 16 hour day at 24° C. for approximately two to six weeks. It is to be understood that seeds of any woody or herbaceous plant species can be used in place of P. taeda L., surface sterilized and aseptically germinated, as above.

### Seedling Inoculation

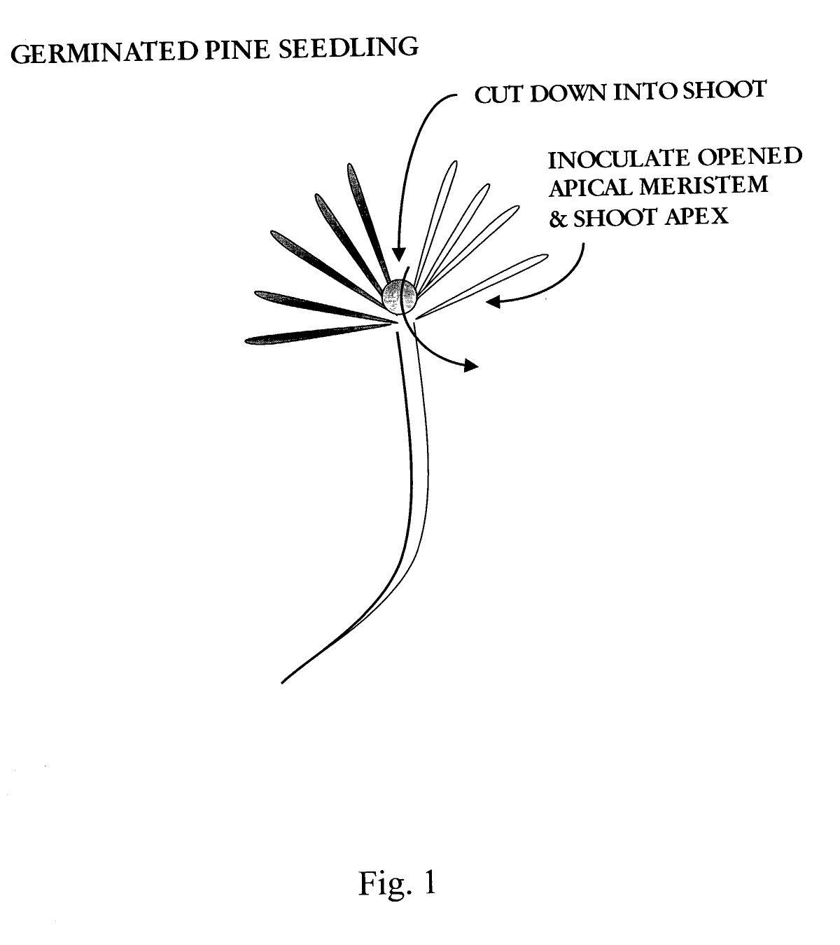

FIG. 1 is a graphical representation of an intact germinated pine seedling and partially illustrates the practice of an embodiment of the disclosed method. Seeds were germinated three to four weeks prior to shoot inoculation. Using a hypodermic needle or small scalpel, an opening (window) was made in the side of the seedling shoot apex that included the apical meristem (FIG. 1). The opened area of the intact germinated seedling shoots were directly inoculated in situ with a virulent strain of A. tumefaciens. Although any strain of A. tumefaciens is adequate, either EHA101 (pGUS3) (Gould et al. 1991) or EHA105 (pSSla.3) (Campbell et al. 1994) were used. The seedlings were co-cultivated with A. tumefaciens for up to 7 days (20° C.) in darkness. A virulence induction solution is used with A. tumefaciens at the time of inoculation to ensure induction of virulence and the transformation process. Acetosyringone and other agents were included to ensure induction of bacterial virulence and gene transfer functions in A. tumefaciens. The cytokinin benzyladenine (BA) was included in the plant culture media (1 mg/l) and in the bacterial induction medium (1 mg/ml) to ensure activation of cell division in the shoot meristem. Cell division activity in inoculated plant tissues appears to be important in Agrobacterium-mediated transformation (Sangwan et al. 1992; Villemont et al. 1997). The protocol used for inoculation is outlined in Table 1.

After 7 days of inoculation, the seedlings were returned to ¼ GD medium for another 7 days if seedlings were selected (see below), or for one to two weeks then transferred to soil if they were not selected.

### Selection

If it is desired to ensure that all A. tumefaciens are killed, inoculated seedlings may be transferred to water agar containing ¼ GD medium (Grosshof & Doy 1972) containing 500-1000 mg/l Clavamox (amoxicillin+clavenate, SmithKline Beecham Veterinary) and the selection agent-kanamycin, 25 mg/l, for two to three weeks to kill the Agrobacterium infection. Seedlings are preferably laid down horizontally on antibiotic containing media so that the inoculated shoot region is exposed to the antibiotic. While selection using kanamycin resistance is preferred, insertion of gene sequences coding for resistances to other antibiotics such as G418, neomycin, hygromycin or chloramphenical or to herbicides such as glyphosate can be used as well as other selectable genes known to those skilled in the art. Additionally, certain additives may be used to enhance successful infection. These include acetosyringone and certain opines such as, but not limited to octapine, nopaline and leucinopine. This selection step overall is optional.

### Transfer to Soil

The inoculated intact germinated seedlings were removed from plates, rinsed to remove agar, dried on paper towels and sprayed with an antifungal mixture containing 125 μl Subdue (DuPont), 0.425 g Benlate in one liter of water. The seedlings were then transferred to an artificial soil media (Metro Mix 200 or 1 peat: 2 vermiculite (course or medium): 1 perlite). Plants in pots were enclosed in a zip-lock bag, watered and monitored. Bags were opened partially until new growth appeared. Once hardened, plants were transferred to the greenhouse. The method is generally described in Table 1.

### Antibiotics

Clavamox® (amoxicillin trihydrate/clavulanate potassium, Veterinary; SmithKline Beecham, Philadelphia Pa.) was used to eliminate A. tumefaciens from the plant tissues. Clavamox® was added to the culture media as a sterile suspension and prepared by dissolving one sterile 250 mg tablet in 5 ml sterile water 10-20 minutes prior to adding to cooled autoclaved media. Kanamycin-HCl (Sigma, St. Louis Mo.) was used for selection of transgenic tissues and cells. Kanamycin was added as an aqueous filter-sterilized solution (Gould et al. 1998).

### Bacteria, Plasmids, Culture & Induction of Virulence

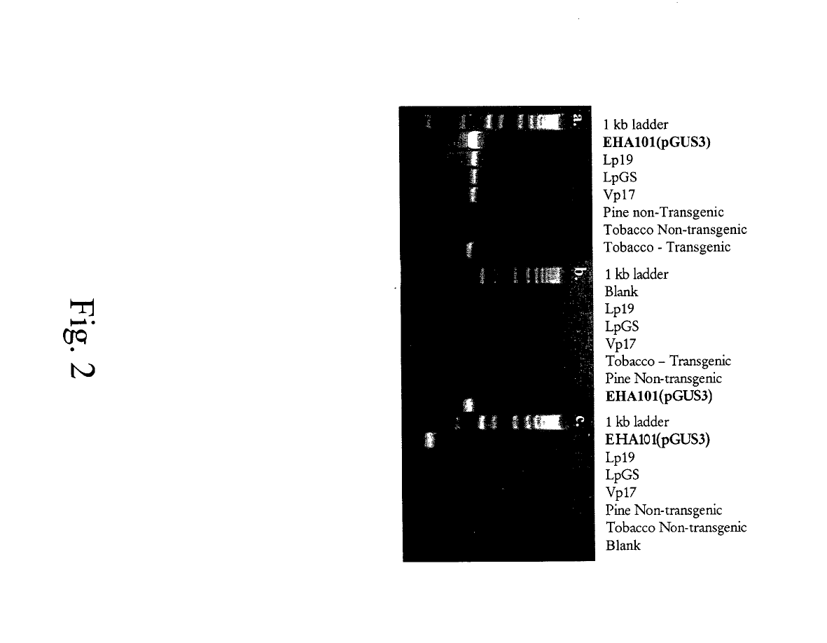

A super-virulent strain of A. tumefaciens harboring the super-virulent pTiBo542 plasmid, had been shown to be effective in transforming cells of Picea abies (Hood et al. 1990). This strain EHA101 (Hood et al. 1986) and the derived strain EHA105 (Hood et al. 1993) were used in this research. The binary vector pGUS3 (FIG. 2a) was used in EHA101 (Gould et al. 1991). The vector pSSLa.3 (FIG. 2b) (Campbell et al. 1994) was used in EHA105. In pGUS3, the expression of uidA (GUS) was driven by an intron-less CaMV 35S promoter; in pSSLa.3, uidA was driven by the Larix laricina RbcS promoter (Campbell et al. 1994). Both vectors carried nos-nptII-nos encoding resistance to kanamycin. The result was a strain of A. tumefaciens which contained the genes for kanamycin resistance and for beta-glucuronidase (GUS). This permitted easy differentiation and detection of transformed tissues.

A. tumefaciens cultures were grown between 22-25° C. for 1-3 days on agar solidified Luria-Bertani medium (LB) (L-3152 Sigma, St. Louis Mo.) or YM (10090-01 Gibco BRL) medium containing the appropriate antibiotics. After growth on plates, the bacteria were scraped from the culture plate and suspended 1:1 (v:v) in 0.5-1 ml filter sterilized virulence induction medium (VIM) containing: acetosyringone, 100 uM (Boulton et al. 1986); octopine, 100 mg/l (Veluthambi et al. 1989); 2% glucose in MES buffer pH=5.4; and 1 mg/ml benzyladenine (BA). Benzyladenine was included to stimulate cell division in the shoot apex and meristem.

### Analyses of Recovered Shoots & Plants

FIG. 2 shows that seedlings were successfully transformed according to one embodiment of the present invention as indicated by PCR amplification data of markers unique to transformed seedlings which are absent in nontransformed seedlings. Intact germinated seedlings, identified as “LpGS”, are transformed after direct inoculation in situ with A. tumefaciens. This ability is a major paradigm shift in the previous methodology; tissue does not need to be excised from the seedling and independently cultured in order to facilitate transformation. Following regeneration of plants, DNA was isolated (Doyle et al. 1987) from young needles. PCR was used to screen for Agrobacterium contamination, as well as for the transferred genes. DNA fragments unique to nptII (1 kb) and uidA (800b) were amplified by PCR in the DNA of regenerated plants: Lp19 (P. taeda), LpGS (P. taeda) and Vp17 (P. virginiana). “LpGS” identifies seedlings that were germinated and directly inoculated in situ with A. tumefaciens. As shown in FIG. 2a (nptII) and 2b (uidA), nptII and uidA, respectively, were expressed by tissues from intact germinated pine seedlings that were directly innoculated with Agrobacterium in situ. Sequences unique to the A. tumefaciens EHA101 & EHA105 virG and picA genes (Zhou et al. 1997; Yusibov et al. 1994) were amplified by PCR in the DNA of the EHA101 (pGUS3) control, but as shown in FIG. 2c, were not amplified in the DNA from regenerated plants Lp19, LpGS, or Vp17, thus demonstrating that the DNA from the putative transgenic pines was not contaminated with Agrobacterium.

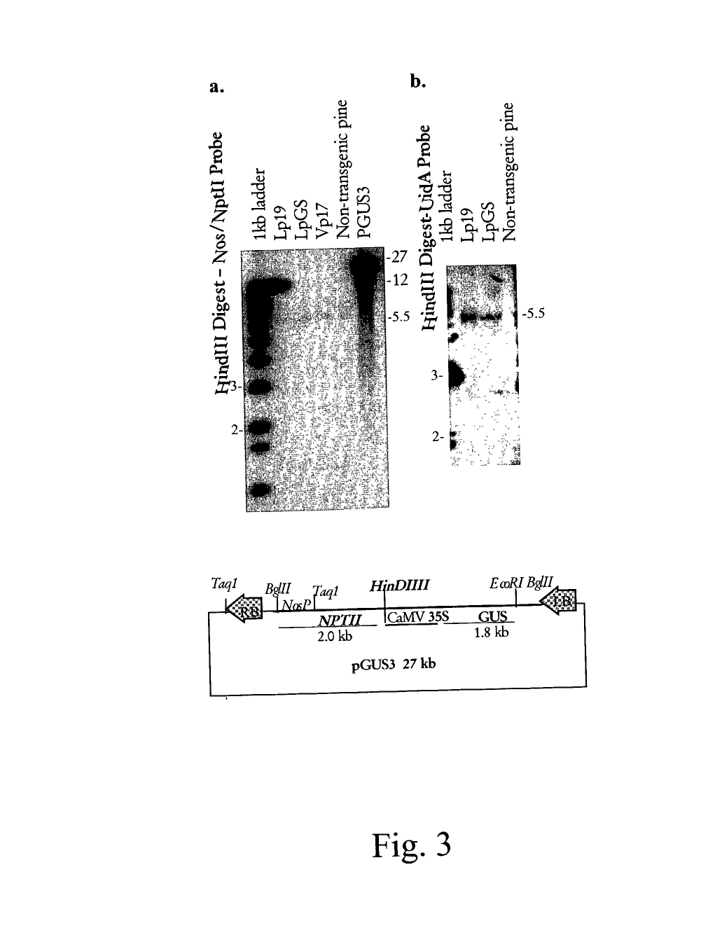

The primers used for PCR amplification for the transferred genes uidA (GUS), nptII (kanamycin resistance), and Agrobacterium genes picA and virG are noted below. The primers used for uidA (GUS) are: forward (5′ TTCGGTGATGATAATCGG CT G) and reverse (5′ GGTATCAGCG CGAAGTCTTA) produced a 1.27 kb fragment within the coding region of the uidA gene. The primers used for nptII: forward (5′ CCCCTCGGTA TCCAATTAGAG) located in the neo coding sequence and reverse (5′ GTGGGCGAAG AACTCCAG) located in the nos promoter, produced a 1 kb fragment spanning the nos promoter and neo coding regions. The primers used for the Agrobacterium chromosomal gene picA are described elsewhere (Yusibov et al. 1994). The primers used for amplification for the pTiBo542 virulence gene virG G: forward 5′ CATAGGCGATCTCCTTAATC and reverse 5′ CGTACTCGAC TGGCAATGAG (Winans 1993). PCR amplification (PCR Master Kit, Boehringer Mannheim) was accomplished according to the manufacturer's directions: 50 pM primers, 100 ng plant DNA template in a 100 ul volume. Cycling conditions were: 96° C., 2 min; Taq and dNPTs added; 94° C., 3 min; 90° C., 30 sec; 55° C., 1 min; 72° C., 3 min for 40 cycles; final extension at 72° C., 10 min. PCR products were separated by electrophoresis through 0.8% agarose gel, stained with ethidium bromide, and visualized by UV light (FIG. 2). Total pine DNA (20 ug) was subjected to either HindIII or Taq1 digestion, separated by gel electrophoresis and blotted to a nylon membrane (Hy-bond Plus, Amersham) under alkaline conditions (Devey et al. 1991). NptII and uidA probes were generated by PCR and labeled with P³² using a random hexamer method (Kit #1004 760, Boherigner Mannheim) as described by the manufacturer. Blots were pre-hybridized and hybridized at 65° C., washed and exposed to film 2-7 days at −70° C. (FIG. 3).

FIG. 3 shows a Southern blot of putative transgenic loblolly pine DNA hybridized with uidA probe indicating that the transferred genes were incorporated into high molecular weight DNA characteristic of stable transformation. Southern blots of DNA isolated from inoculated intact germinated seedlings, identified by “LpGS”, were used to determine the efficacy of the direct inoculation method of intact germinated plants in situ of the present invention. Gel blots of HindIII digested DNA from Lp19, LpGS, Vp17, P. taeda L. control, and pGUS3, were hybridized with P³² labeled uidA. The 27 kb pGUS3 carried a single HindIII site and produced the expected 27 kb fragment upon digestion and hybridization with either uidA or nos-npt probe. The HindIII-digested DNA of Lp19 produced 4-5, 8 and 13 kb fragments with homology to the nos-npt probe. Upon longer exposure, a similar nos-npt 13 kb fragment was also apparent in the DNA of LpGS. The HindIII-digested DNA of the pGUS3 vector produced two bands of 8 kb and 27 kb with homology to uidA (FIG. 3a). As shown in FIG. 3b, when probed with uidA, the HindIII digested DNA from LpGS produced 5.5 kb fragments with homology to uidA. Neither uidA nor nptII probes detected homologous DNA in Vp17 or in the DNA of non-transgenic pine. The restriction patterns produced in the DNA of Lp19 and LpGS were similar to each other, but not similar to the restriction pattern of the transforming plasmid pGUS3, indicating that the transferred genes are not present as plasmid or as Agrobacterium contamination.

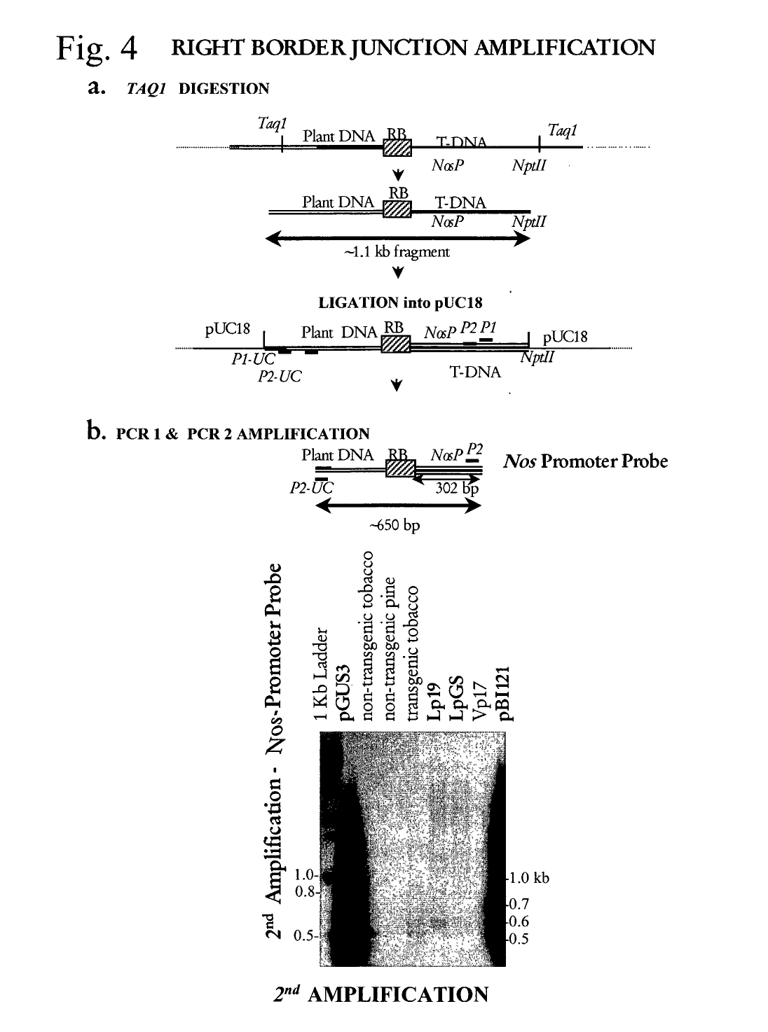

FIG. 4 shows PCR amplification data of the plant border junction to show that intact germinated seedlings were transformed by Agrobacterium by direct inoculation in situ. To verify that the T-DNA of Agrobacterium integrated into the plant genomic DNA, identification of the junction sequences spanning the region between the nos-promoter/T-DNA border and plant genomic DNA were detected using a method originally developed to isolate and amplify fragments for sequencing (Zhou et al. 1997). Briefly, Taq1 digested plant or vector DNA was ligated into an Acc1-digested pUC18 plasmid (FIG. 4a). The ligation mixture was subjected to two rounds of PCR amplification using nested primers having homology to the nos promoter (nos-P) and to a sequence within the pUC universal cloning site. The two consecutive PCR reactions used different but nested sets of primers. The primers for the first set, identified in FIG. 4a as “P1-UC” and “P1”, respectively, are: forward (5′-CGAATAGCCTCTCCACCCAAGC GGCCG-3′) and reverse (5′-TCGTATG TTGTGTGGAATTGGAGCGG-3′). The second set of primers which were nested to the primers of the first PCR amplification, are: forward (5′-ATTGGAT ACCGAG GGG-3′) and reverse (5′-AACAGCTATGACCATG-3′). These primers are identified in FIG. 4a, respectively, as “P2-UC” and “P2”. Fragments were amplified above the detection limit in the second PCR amplification. As shown in FIG. 4b, border fragments of ˜600 b with homology to the nos-promoter probe were detected in the DNA of loblolly pine, shown as LpGS, directly inoculated with Agrobacterium as a germinated seedling. The final PCR products were separated on an agarose gel, the gel blotted to a membrane and hybridized with P³² labeled nos promoter probe (FIG. 4b). Blots were washed and exposed to film. The fragments of ˜600 b that would contain the nos/nptII and plant border junction sequences are seen in the DNA of (l to r): transgenic tobacco, Lp19 and LpGS. (FIG. 4b) These results indicate presence of a T-DNA right border fragment incorporated into plant genomic DNA in these intact plants that were directly inoculated with A. tumefaciens in situ.

In summary, the invention discloses a direct Agrobacterium inoculation transformation method of intact seedlings to recover transgenic plants. Genetic fidelity is most closely maintained in the meristems and the germline of plants, and inoculated plant regeneration is straight-forward and simple. Seedlings are directly inoculated with a virulent strain of Agrobacterium tumefaciens, subsequently subjected to selection, and then generated directly into plants, thus making the process genotype-independent. Tissues do not pass through a dedifferentiation step to callus, and plant regeneration is not dependent on shoot organogenesis or somatic embryogenesis nor is it limited to regenerable genotypes.

Various basics of the invention have been explained herein. The various techniques and devices disclosed represent a portion of that which those skilled in the art would readily understand from the teachings of this application. Details for the implementation thereof can be added by those with ordinary skill in the art. The accompanying figures may contain additional information not specifically discussed in the text and such information may be described without adding new subject matter. Additionally, various combinations and permutations of all elements or applications can be created and presented. All can be done to optimize performance in a specific application.

The various steps described herein can be combined with other steps and they can occur in a variety of sequences unless otherwise specifically limited. These various steps can be interlineated with the stated steps, and the stated steps can be split into multiple steps. Unless the context requires otherwise, the word “comprise” or variations such as “comprises” or “comprising”, should be understood to imply the inclusion of a stated element or step or group of elements or steps but not the exclusion of any other element or step or group of elements or steps.

Further, any references mentioned in the application for this patent as well as all references listed in any list of references filed with the application are hereby incorporated by reference. However, to the extent statements might be considered inconsistent with the patenting of this invention, such statements are expressly not to be considered as made by the applicant(s).

### REFERENCES

1. U.S. Pat. No. 5,164,310, Method for transforming plants via the shoot apex, Nov. 17, 1992, Inventors: Smith; Roberta H. (Hearne, Tex.); Gould; Jean H. (Bryan, Tex.); Ulian; Eugenio (College Station, Tex.), Assignee: The Texas A&M University System (College Station, Tex.)

2. Boulton G, Nester E, Gordon M: Plant phenolic compounds induce expression of the Agrobacterium tumefaciens loci needed for virulence. Science 232: 983-985 (1986).

3. Campbell M A, Neale D B, Harvie P, Hutchison K W: Tissue-specific and light regulation of a larch ribulose-1, 5-bisphosphate carboxylase promoter in transgenic tobacco. Can J For Res 24: 1689-1693 (1994).

4. Chang S, Sen S, McKinley C, Aimers-Halliday J, Newton R J: Clonal propagation of Virginia Pine (Pinus Virginia Mill.) by organogenesis. Plant Cell Rep 10: 131-134 (1991).

5. Devey M, Jermstad K D, Tauer C G, Neale D B: Inheritance of RFLP loci in a loblolly pine three-generation pedigree. Theor Appl Genet 83: 238-242 (1991).

6. Dong J, Teng W, Buchholtz W, Hall T: Agrobacterium-mediated transformation of javanica rice. Mol Breed 9: 1-10 (1996).

7. Doyle J J, Doyle J L: A rapid DNA isolation procedure for small quantities of fresh leaf tissue. Biotech Bull 19: 11-15 (1987).

8. Ellis D, Roberts D, Sutton B, Lazaroff W, Webb D, Flinn B: Transformation of white spruce and other conifer species by Agrobacterium tumefaciens. Plant Cell Rept 8: 16-20 (1989).

9. Gould J H, Devey M E, Hasegawa O, Ulian E C, Peterson G, Smith R H: Transformation of Zea mays L. using Agrobacterium tumefaciens and the shoot apex. Plant Physiol 95: 426-434 (1991).

10. Gould J H, Magallanes M E: Adaptation of Shoot Apex Agrobacterium Inoculation & Culture to Cotton Transformation. Plant Mol. Biol. Rept. 16: 284-289 (1998).

11. Gresshof P M, Doy C H: Development and differentiation of haploid Lycopersicon esculentum L., (tomato). Planta 107: 161-170 (1972).

12. Hirochika H, Sugimoto K, Otsuki Y, Tsugawa H and Kanda M: Retrotransposons of rice involved in mutations induced by tissue culture. Proc Natl Acad Sci USA 93: 7783-7788 (1996).

13. Hood E E, Helmer G L, Fraley R T, Chilton M-D. 1986. The hyper-virulence of Agrobacterium tumefaciens A281 is encoded in a region of pTiBo542 outside of T-DNA. J Bacteriol. 168:1283-1290.

14. Hood E E, Clapham D H, Ekberg I, Johannson T: T-DNA presence and opine production in tumors of Picea abies (L.) Karst. induced by Agrobacterium tumefaciens A281. Plant Mol Biol 14: 111-117 (1990).

15. Hood E E, Gelvin S B, Melchers L S, Hoekema A: New Agrobacterium helper plasmids for gene transfer to plants. Trans Res 2: 208-218 (1993).

16. Huang Y, Tauer C G: Integrative transformation of Pinus taeda L. and P. echinata Mill, Agrobacterium tumefaciens. Forest Genet 1: 23-31 (1994).

17. Li R, Stelly D, Trolinder N: Cytogenetic abnormalities in cotton (Gossypium hirsutum L.) cell cultures. Genome 32: 1128-1134 (1989).

18. Morel G, Martin C, Muller J F: La guerison de pommes de terre atteintes de maladies a virus. Annu Physiol Veg 10: 113-139 (1968).

19. Murashige T: Plant propagation through tissue cultures. Annu Rev Plant Physiol 25: 135-166 (1974).

20. Oard J H, Linscombe S D, Braverman M P, Jodari F, Blouin D C, Leech M, Kohli A, Vain P, Cooley J C, Christou P: Development, field evaluation, and agronomic performance of transgenic herbicide resistant rice. Mol Breed 2: 359-368 (1996).

21. Sangwan R S, Bourgeois Y, Brown S, Vasseur G, Sangwan-Norreel B: Characterization of competent cells and early events of Agrobacterium-mediated genetic transformation in Arabidopsis thaliana. Planta 188: 439-456 (1992).

22. Sederoff R J, Stomp A, Chilton W S, Moore L W. 1986. Gene transfer into loblolly pine by Agrobacterium tumefaciens. Bio/Technology 4:647-649.

23. Sen S, Magallanes-Cedeno M E, Kamps R H, McKinley C R, Newton R J: In vitro micropropagation of afghan pine. Can J For Res 24: 1248-1252 (1993).

24. Smith D R: The role of in vitro methods in pine plantation establishment: the lesson from New Zealand. Plant Tissue Culture and Biotech 3: 63-67 (1997).

25. Stomp A M: Histochemical localization of beta-glucuronidase. In: Gallagher S R (ed) GUS Protocols. Academic Press Inc. NY pp. 103-114 (1992).

26. Svitashev S, Ananiev E, Pawlowski W P, Somers D A: Association of transgene integration sites with chromosome rearrangements in hexaploid oat. TAG 100: 872-880 (2000).

27. Tang W, Sederoff R, Whetten R: Regeneration of transgenic loblolly pine (Pinus taeda L.) from zygotic embryos of loblolly pine. Planta 213:981-989.

28. Veluthambi K, Krishnan M, Gould J H, Smith R H, Gelvin S B: Opines stimulate the induction of the VIR genes of the Agrobacterium tumefaciens Ti plasmid. J Bacteriol 171:3696-3703(1989).

29. Villemont E, Dubois F, Sangwan R S, Vasseur G, Bourgeois Y, and Sangwan-Norreel BS: Role of the host cell cycle in the Agrobacterium-mediated genetic transformation of petunia-evidence of an S-phase control mechanism for T-DNA transfer. Planta 201:160-172 (1997).

30. Walter C, Grace L J, Wagner A, White D W R, Walden A R, Donaldson S S, Hinton H, Gardener R C, Smith D R: Stable transformation and regeneration of transgenic plants of Pinus radiata, Don. Plant Cell Rept 17:460-468 (1998).

31. Wenck A R, Quinn M, Whetten R, Pullman G, Sederoff R: High-efficiency Agrobacterium-mediated transformation of Norway spruce (Picea abies) and loblolly pine (Pinus taeda). Plant Mol Biol 39:407-416 (1999).

32. Winans S: A. tumefaciens plasmid pTi pBo542 virG gene. GenBank Database X62885S70493 (1993).

33. Zhou Y, Newton R J, Gould J H: A simple method for identifying plant/T-DNA junction sequences resulting from Agrobacterium-mediated DNA transformation. Plant Mol Biol Rept 15: 246-254 (1997).

34. Yusibov V M, Steck T R, Gupta V, Gelvin S B: Association of single-stranded transferred DNA from Agrobacterium tumefaciens with tobacco cells. Proc Natl Acad Sci USA 91: 299-2998 (1994).

TABLE 1
INOCULATION & CULTURE SCHEDULE

| Procedure | Time | Media & Components |
|---|---|---|
| SEEDLINGS | 3-4 | Germination. Seeds germinated 3-4 weeks |
| | weeks | in water agar plates. |
| BACTERIA | 1-3 | A. tumefaciens EHA101, or EHA105, grown |
| | days | on LB or YM agar solidified medium |
| | | (Sigma, St Louis Mo), containing |
| | | appropriate antibiotics, 1-3 days at |
| | | 22-26° C. Induction of Bacterial Virulence: |
| | | 75 mM MES pH = 5.4; acetosyringone, |
| | | 100 uM; glucose, 2%; octopine, 100 mg/l, |
| | | benzyladenine 1 mg/ml at 22-26° C. |
| INOCULATION | 7 days | Seedling shoots wounded and inoculated |
| | | with Agrobacterium suspended in virulence |
| | | induction medium. Return entire seedling |
| | | to water agar plates or ¼ GD media in |
| | | plates. Co-cultivate at 20-24° C. for 7 days |
| | | in dark or under low light. |
| CULTURE | 7 days | Return seedling to light and room temper- |
| | | ature conditions. If Selection is not used, |
| | | wait 1-2 weeks then transfer to soil. |
| SELECTION | 3-4 | Antibiotic selection (optional). Lay seedling |
| | weeks | down with the shoot region touching media |
| | | comprised of ¼ GD + kanamycin 25 mg/l + |
| | | Clavamox 500 mg/l + benzyladenine 1 mg/l. |
| | 2-3 | Recovery from Selection (optional). Move |
| | weeks | seedling from selection. Lay seedling down |
| | | onto ¼ GD. |
| SOIL | | Transfer seedlings to an artificial soil media |
| | | in 2-4″ pots after spraying plants with |
| | | Subdue mixture & enclose in zip-lock plastic |
| | | bag to harden-off. Retain in culture room |
| | | until new growth appears, then transfer to |
| | | greenhouse. |
| TOTAL TIME | 5.5-7.5 | If Selection & Recovery steps are not used |
| | weeks | |
| | 11-14 | If Selection and Recovery steps are included |
| | weeks | |

The procedure describes use with loblolly pine seedlings. Total germination time, as well as time needed for selection & recovery, can vary for seeds/seedlings of other plant species.

1. A method of transforming intact plant tissue comprising:
(1) germinating an intact plant seedling having a shoot apex, and
(2) directly inoculating said shoot apex of said germinated intact plant seedling with Agrobacterium tumefaciens to transform said seedling.

2. The method of claim 1 wherein said intact plant seedling is germinated for a period no longer than about 6 weeks.

3. The method of claim 1 wherein said intact plant seedling is a gymnosperm.

4. The method of claim 1 wherein said method further comprises selecting inoculated germinated intact plant seedling.

5. The method of claim 4 wherein said inoculated germinated intact plant seedling is selected with at least one antibiotic.

6. The method of claim 1 wherein said Agrobacterium tumefaciens is a super-virulent strain.

7. The method of claim 1 wherein said method further comprises rooting said inoculated germinated intact plant seedling.

8. The method of claim 1 wherein said germinated intact plant seedling is a gymnosperm.

9. The method of claim 8 wherein said germinated intact plant seedling is a conifer.

10. The method of claim 9 wherein said germinated intact plant seedling is a pine.

11. The method of claim 1 wherein said germinated intact plant seedling is an angiosperm.

12. The method of claim 11 wherein said intact plant seedling is a monocot.

13. The method of claim 11 wherein said intact plant seedling is a dicot.

14. The method of claim 1 where the method is completed for a period no longer than 8 weeks.

15. The method of claim 4 wherein the method is completed for a period no longer than 15 weeks.

16. A method of transforming intact plant tissue comprising:
(1) germinating an intact plant seedling having a shoot apex;
(2) directly inoculating said shoot apex of said germinated intact plant seedling with Agrobacterium tumefaciens, wherein at least one cell of said germinated intact plant seedling is transformed; and
(3) selecting said inoculated germinated intact plant seedling.

17. The method of claim 16 wherein said inoculated germinated intact plant seedling is selected with at least one antibiotic.

18. The method of claim 16 wherein the age of said germinated intact plant seedling is between about 3 to about 6 weeks.

19. The method of claim 16 wherein said method further comprises rooting said inoculated germinated intact plant seedling.

20. The method of claim 16 wherein said intact plant seedling is a gymnosperm.

21. The method of claim 20 wherein said intact plant seedling is a conifer.

22. The method of claim 21 wherein said intact plant seedling is a pine.

23. The method of claim 16 wherein said intact plant seedling is an angiosperm.

24. The method of claim 23 wherein said intact plant seedling is a monocot.

25. The method of claim 23 wherein said intact plant seedling is a dicot.

26. The method of claim 16 wherein said method is completed in no longer than 15 weeks.

27. A method of transforming intact plant tissue comprising:
(1) germinating an intact plant seedling having a shoot apex;
(2) opening said shoot apex of said germinated intact plant seedling;
(3) directly inoculating said shoot apex of said germinated intact plant seedling with Agrobacterium tumefaciens, wherein at least one cell of said shoot apex is transformed; and
(4) selecting said inoculated germinated intact plant seedling.

28. The method of claim 27 wherein said opening of said germinated intact plant seedling is at the shoot apex.

29. The method of claim 27 wherein said step of opening said germinated intact plant plant seedling is at an apical meristem of said shoot apex.
